(19) United States
(12) Patent Application Publication
(10) Pub. No.: US 2008/0058559 A1
(43) Pub. Date: Mar. 6, 2008

(54) PREPARATION OF HIGH ASSAY DECABROMODIPHENYL OXIDE

(75) Inventors: Saadat Hussain, Baton Rouge, LA (US); Arthur G. Mack, Prairieville, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/843,759

(22) Filed: Aug. 23, 2007

Related U.S. Application Data
(60) Provisional application No. 60/823,834, filed on Aug. 29, 2006.

(51) Int. Cl. C07C 41/22

(52) U.S. Cl. 568/639

(57) ABSTRACT

Improvements are described in process technology for producing reaction-derived decabromodiphenyl oxide of high purity from (i) diphenyl oxide and/or partially brominated diphenyl oxide and (ii) bromine under specified conditions including substantially concurrently reducing the content of hydrogen bromide present in the reactor sufficiently so that reaction-derived decabromodiphenyl oxide of high purity is formed without requiring procedures such as recrystallization or chromatography to remove lower brominated species, especially nonabromodiphenyl oxide.

### REFERENCE TO RELATED APPLICATION

This application claims the benefit and priority of U.S. Provisional Application No. 60/823,834, filed Aug. 29, 2006, the disclosure of which is incorporated herein by reference.

### TECHNICAL FIELD

This invention relates to improvements in the preparation of high assay decabromodiphenyl oxide products of high purity.

### BACKGROUND

Decabromodiphenyl oxide (DBDPO) is a time-proven flame retardant for use in many flammable macromolecular materials, e.g. thermoplastics, thermosets, cellulosic materials and back coating applications.

DBDPO is presently sold as a powder derived from the bromination of diphenyl oxide (DPO) or a partially brominated DPO containing an average of about 0.7 bromine atom per molecule of DPO. Such bromination is conducted in excess bromine and in the presence of a bromination catalyst, usually AlCl₃. The operation is typically conducted at 177° F. (ca. 80.5° C.) with a 2-3 hour feed time. The powdered products are not 100% DBDPO, but rather are mixtures that contain up to about 98% DBDPO and about 1.5%, or a little more, of nonabromodiphenyl oxide co-product. As a partially brominated product, this amount of nonabromodiphenyl oxide is considered problematic by some environmental entities.

It would therefore be desirable to provide process technology enabling preparation of DBDPO products of higher purity, such as products comprising (A) at least 99% of DBDPO and (B) nonabromodiphenyl oxide in an amount not exceeding 0.5%, preferably not exceeding 0.3%, and still more preferably, not exceeding about 0.1%. It would be especially desirable if such technology could produce DBDPO products comprising (A) at least 99.5% of DBDPO and (B) nonabromodiphenyl oxide in an amount not exceeding 0.5%, preferably not exceeding 0.3%, and still more preferably, not exceeding about 0.1%.

### BRIEF DESCRIPTION OF THE DRAWING

FIG. 1 is a vertical cross-sectional view of a jet mixer injection device suitable for effecting improvements pursuant to this invention.

### BRIEF SUMMARY OF THE INVENTION

Commonly-owned copending U.S. Application No. 60/823,811, filed on Aug. 29, 2006, and entitled “Preparation of High-Assay Decabromodiphenyl Oxide”, all disclosure of which is incorporated herein by reference, describes process technology capable of producing DBDPO products of high purity, such as products comprising (A) at least 99% of DBDPO and (B) nonabromodiphenyl oxide in an amount not exceeding 0.5%, preferably not exceeding 0.3%, and still more preferably, not exceeding about 0.1%. Indeed, that process enables production of DBDPO products comprising (A) at least 99.5% of DBDPO and (B) nonabromodiphenyl oxide in an amount not exceeding 0.5%, preferably not exceeding 0.3%, and still more preferably, not exceeding about 0.1%. One embodiment of the process technology of the foregoing application comprises feeding diphenyl oxide and/or partially brominated diphenyl oxide substantially continuously over a period in the range of about 2 to about 12 hours into a reactor containing a refluxing reaction mixture comprising (i) an excess of bromine and (ii) a catalytic quantity of Lewis acid bromination catalyst, and substantially concurrently removing hydrogen bromide coproduct from the reactor in a sufficient amount to form a reaction-derived decabromodiphenyl oxide product of high purity. Another embodiment of the process technology of the foregoing application comprises maintaining a substantially continuous, inversely related time-temperature feed of diphenyl oxide and/or partially brominated diphenyl oxide to a reactor containing a refluxing reaction mixture comprising (i) an excess of bromine and (ii) a catalytic quantity of Lewis acid bromination catalyst, and substantially concurrently reducing the concentration of hydrogen bromide coproduct dissolved in the liquid phase of the reaction mixture so that reaction-derived decabromodiphenyl oxide product of high purity is formed.

This invention provides improvements in the process technology described and claimed in the foregoing application.

One of the improvements in each of the embodiments of the foregoing application comprises separately and substantially concurrently feeding (i) bromine and (ii) said diphenyl oxide and/or partially brominated diphenyl oxide into the reactor of the process. Separate and concurrent feeding is sometimes referred to in the art as “co-feeding”. Stated another way, this improvement comprises concurrently feeding at least two separate feeds into the reaction zone, one of the separate feeds being bromine and the other, or another, of the separate feeds being or comprising the diphenyl oxide and/or partially brominated diphenyl oxide.

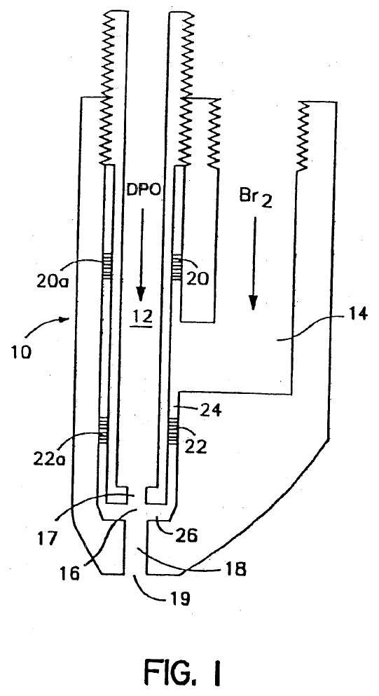

Another improvement in each of the embodiments of the foregoing application is to mix the separate feeds of (i) and (ii) together in a small chamber for a period of less than about 2 seconds prior to injecting the mixture into the body of the refluxing reaction mixture contained in the reactor. In short, a single mixture is fed into the reactor but such single mixture is formed in a particular way. In this improvement, the small chamber serves as a mixing zone and as a small reaction zone in which refluxing need not occur, and from which in the period of less than about 2 seconds, the mixed and initially reacting (i) and (ii) are injected into the body of the refluxing reaction mixture in the reactor. FIG. 1 illustrates a preferred injection device containing a small chamber serving the functions just described. Thus, when using an injection device containing a small chamber such as just described, the improvement comprises separately and concurrently feeding (i) liquid bromine and (ii) diphenyl oxide and/or partially brominated diphenyl oxide into a small mixing/initial reaction zone, and feeding the resultant mixture into the reactor.

Thus, in one of its embodiments, this invention provides, in a process for preparing reaction-derived decabromodiphenyl oxide of high purity, which process comprises feeding diphenyl oxide and/or partially brominated diphenyl oxide into a reactor containing a refluxing reaction mixture comprising (i) an excess of bromine and (ii) a catalytic quantity of Lewis acid bromination catalyst, and substantially concurrently removing hydrogen bromide coproduct from the reactor in a sufficient amount to form a reaction-derived decabromodiphenyl oxide product of high purity, the improvement which comprises:
A) substantially concurrently feeding at least two separate feeds into said reactor, one of said separate feeds being bromine and the other, or another, of said separate feeds comprising said diphenyl oxide and/or partially brominated diphenyl oxide; or
B) substantially concurrently feeding at least two separate feeds into a small mixing zone, one of said separate feeds being bromine and the other, or another, of said separate feeds comprising said diphenyl oxide and/or partially brominated diphenyl oxide, followed within 2 seconds or less by feeding the resultant mixture into said reactor.

Among advantages deemed to result from the feeding improvements of this invention are the following:
1) As long as the separately and substantially concurrently fed components (i) and (ii) are suitably mixed together in the reactor, the accumulation of bromine within the particles or crystals of the reaction-derived decabromodiphenyl oxide product is reduced, if not eliminated.
2) As a consequence of 1) above, the difficulty and expense of utilizing procedures to remove bromine from the particles or crystals of the reaction-derived decabromodiphenyl oxide product is avoided, or at least minimized.
3) The filtration rate of the reaction-derived decabromodiphenyl oxide product is improved.

The above and other embodiments and features of this invention will be still further apparent from the ensuing description and appended claims.

### FURTHER DETAILED DESCRIPTION

In accordance with various embodiments of this invention there is provided aprocess for preparing a reaction-derived decabromodiphenyl oxide product of high purity. The process comprises separately and substantially concurrently feeding (i) diphenyl oxide (DPO) and/or underbrominated DPO and (ii) bromine, into a reactor containing a refluxing reaction mixture formed from (i) and (ii), which reaction mixture contains Lewis acid bromination catalyst and has a liquid phase which includes liquid bromine, and concurrently reducing the content of coproduct hydrogen bromide from the reaction mixture so that a reaction-derived decabromodiphenyl oxide product of high purity is formed. The amount of bromine being fed to the reactor is in excess of the amount of (i) being fed to the reactor. Such excess amount is preferably in the range of about 50 to about 150 mole percent more than the amount theoretically required to perbrominate the feed of (i).

As noted above an improvement of this invention comprises separately and substantially concurrently feeding (i) diphenyl oxide (DPO) and/or underbrominated DPO and (ii) bromine, into a small chamber serving as a mixing zone and as a small reaction zone in which refluxing need not occur, and from which in the period of less than about 2 seconds, the mixed and initially reacting (i) and (ii) above are injected into the body of the refluxing reaction mixture present in the reactor. FIG. 1 illustrates in vertical cross-sectional view a jet mixer injection device well suited for use in practicing this improvement in the process. The device, generally designated by the numeral 10, provides a longitudinal, axially directed conduit 12 through which the liquid diphenyl oxide and/or partially brominated diphenyl oxide (collectively referred to in FIG. 1 as “DPO”) flows. Conduit 14 carries the bromine to an annular space 24 which surrounds conduit 12. Spacers 20, 20a, 22 and 22a locate and hold conduit 14 in position with respect to annular space 24. At the lowermost extent of annular space 24 there is radial conduit 26 which directs the bromine flow in an inward and radial direction with respect to the long axis of conduit 12. Adjacent liquid discharge port 17 and radial conduit 26 is impingement chamber 16. Downstream from impingement chamber 16 is a small mixing chamber 18 and mixture discharge port 19.

In operation, bromine flows through conduit 14, annular space 24 and radial conduit 26 to reach impingement chamber 16. At impingement chamber 16 the bromine is traveling in an inward and radial direction. The diphenyl oxide and/or partially brominated diphenyl oxide flows down conduit 12 and through discharge port 17 in an axial direction with respect to impingement chamber 16. The thus flowing diphenyl oxide and/or partially brominated diphenyl intersects and impinges perpendicularly with the flowing bromine from radial conduit 26. Subsequent to the impingement, the resulting mix flows into mixing chamber 18 and is then discharged with velocity as a stream from the device.

In a typical device, the height of radial conduit 26 is about 0.635 cm (¼ inch) while mixing chamber 18 is about 0.80 cm ( 5/16 inch) in diameter and about 1.9 cm (¾ inch) in length. The mixer dimensions, which determine the velocity of the stream from the mixer and the residency time of the mixture formed from (i) bromine and (ii) diphenyl oxide and/or partially brominated diphenyl oxide in the mixing chamber, can be conventionally determined.

The processes of this invention can be conducted as a batch process or as a continuous basis. In general, the duration of the feeding period in a batch process is inversely related to the temperature at which the refluxing is occurring. In other words, the higher the temperature, the shorter can be the feed time. When operating as a continuous process, the duration of the average residence time in the reactor is inversely related to the temperature at which the refluxing is occurring.

As used herein including the claims:
1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenyl oxide from decabromodiphenyl oxide.
2) The term “high purity” means that the reaction-derived DBDPO product comprises more than 99% of DBDPO and nonabromodiphenyl oxide in an amount of less than 1% with, if any, a trace of octabromodiphenyl oxide. Preferably the process forms a reaction-derived product which comprises (A) at least 99.5% of DBDPO and (B) nonabromodiphenyl oxide in an amount not exceeding 0.5%, preferably not exceeding 0.3%, and still more preferably, not exceeding about 0.1%.
3) The terms “substantially concurrently” and “concurrent” as regards the feeding of (i) and (ii) means that the feeding of (i) is taking place at the same time or at substantially the same time that the feeding of (ii) is taking place and vice versa. However, the feeds need not start at precisely the same moment in time, nor must they cease at the same moment in time. In addition, either or both of the feeds of (i) and (ii) can be interrupted as long a the interruptions are short enough in duration as not to preclude the formation of reaction-derived decabromodiphenyl oxide product of high purity.

For the purposes of this invention, unless otherwise indicated, the % values given for DBDPO and nonabromodiphenyl oxide are to be understood as being the area % values that are derived from gas chromatography analysis. A procedure for conducting such analyses is presented hereinafter.

Another embodiment is a process of preparing reaction-derived decabromodiphenyl oxide of high purity, which process comprises maintaining separate and concurrent, inversely related time-temperature feeds of (i) diphenyl oxide (DPO) and/or partially brominated DPO and (ii) bromine to a reactor containing a refluxing reaction mixture comprising an excess of bromine containing Lewis acid bromination catalyst, and substantially concurrently reducing the concentration of hydrogen bromide coproduct dissolved in the liquid phase of the reaction mixture so that reaction-derived DBDPO product of high purity is formed. In this embodiment the higher the bromination reaction temperature, the shorter is the time duration of the feeds, and the lower is the pressure in the reactor.

Accordingly, the length of the feeding period in a batch operation and the average residence time in a continuous operation is temperature dependent. In this connection, and without being bound by theory, it appears that this temperature dependence effect is related to the time required to reach the desired equilibrium state described above. Thus, in carrying out a process of this invention, if the temperature-dependent period has not already been determined for the particular operation, a few laboratory experiments should be conducted for optimization purposes. It is to be noted that at any given temperature use of a higher concentration of catalyst may enable the reaction time to be shortened to some extent, provided that the hydrogen bromide concentration in the liquid phase of the reaction mixture is kept to a minimum or at least low enough as not to prevent preparation of reaction-derived DBDPO of high purity.

Generally speaking, from the viewpoint of productivity and plant throughput in a batch operation, the shorter the separate and concurrent feed periods used, the better. But pursuant to this invention the separate and concurrent feed periods used should be sufficiently long at the reaction temperature being used to enable the desired equilibrium state to be reached whereby the reaction-derived product is a high purity product.

Therefore, depending on the temperature at which the bromination is occurring in a batch process, the separate and concurrent feeds of DPO and/or partially brominated DPO should occur during a sufficiently long period in the range of about 2 to about 12 hours, and preferably in the range of about 4 to about 10 hours to reach the desired equilibrium state. When operating at a plant scale this period of time in part represents a compromise between rate of reactor throughput and desire for as slow a feed as is practicable for achieving the desired product purity. Thus, the duration of the substantially continuous feed should be a period of time that is prolonged yet consistent with achieving an economically acceptable plant throughput. The use of a slow feed is desirable as it provides a longer period of time for a given quantity of DPO or partially brominated DPO to reach the decabromodiphenyl oxide stage before significant precipitation of nonabromodiphenyl oxide encased in decabromodiphenyl oxide particles takes place.

In practicing a process of this invention it is important to reduce the content of hydrogen bromide present in the reactor. Among various ways of achieving such reduction in the amount of hydrogen bromide present in the reactor are the following:

A combination of vigorous refluxing of the bromine in the reactor, withdrawal of the hydrogen bromide vapor phase from the reactor, and efficient condensation of bromine vapors being withdrawn with the hydrogen bromide is desirable and is preferably utilized.

Use of a fractionation column to effectively separate as much HBr from the bromine in the column as feasible. In this way the bromine returning to the reactor carries less, if any, HBr back into the reactor. The fractionation column can be a packed column or it can be free of packing, and should be designed to effect an efficient separation of HBr from bromine.

An inert gas purge of the reactor (e.g., with argon, neon, or preferably nitrogen) to carry away HBr is useful.

Use of bromine in the vapor state as a stripping gas. Besides carrying away HBr, the use of bromine vapors is a way of introducing more heat into the reactor and thereby contributing to more vigorous refluxing within the system.

Operation at atmospheric, subatmospheric or superatmospheric pressures to enable a refluxing condition of the reaction mixture at the selected process temperature.

Since the bromination is conducted in excess refluxing bromine, the reactor is of course equipped with a reflux condenser and preferably a reflux fractionation column. This should be designed to return to the reaction as little HBr in the condensed bromine as is technically and economically feasible under the circumstances.

Another way deemed to reduce the content of hydrogen bromide present in the reactor comprises reoxidizing hydrogen bromide dissolved in the reaction mixture to thereby convert the hydrogen bromide into bromine, for example, by use of a suitable oxidant that converts hydrogen bromide into bromine without destroying the bromination catalyst.

In all cases, the hydrogen bromide leaving the reaction system is preferably recovered for use or sale. Recovery can be achieved by use of a suitable scrubbing system using one or more aqueous liquid scrubbers such as water whereby hydrobromic acid is formed, or dilute base solution such as a solution of NaOH or KOH whereby a solution of sodium bromide or potassium bromide is formed from which such bromide salts can readily be recovered.

The relationship between bromination reaction temperature and pressure under which the bromination is being operated is worthy of comment. Ideally it is desirable to operate at as high a temperature as possible and as low a pressure as possible to adequately reduce the HBr concentration in the bromine, because in this way more HBr is removed from the reactor. Sampling a refluxing bromination reaction mixture of this type in order to assay the percentage of HBr dissolved in the Br₂ at any given time is not deemed feasible when using ordinary laboratory or plant equipment. Such sampling requires special equipment such as built-in stationary probes to periodically remove representative samples of the reaction mixture from the reactor. Thus when using ordinary plant equipment, operation at maximum temperature and minimum pressure is desirable as a way of reducing the HBr concentration in the bromine. However, maintaining a high reaction temperature in such a reaction system is not as easy as it might appear. For one thing, considerable heat input is required to the reaction mixture, and this can impose limitations in existing plant equipment. Consequently, in most cases it is desirable when operating on a commercial scale to conduct the reaction at a mildly elevated pressure (e.g., in the range of about 5 to about 20 psig (ca. 1.36×10⁵ to 2.39×10⁵ Pa)), and having the temperature high enough to effect vigorous refluxing to thereby keep the HBr concentration in the bromine low as more HBr is removed from the reactor.

This invention enables the preparation of highly pure DBDPO products that are derived from the bromination of diphenyl oxide and/or partially brominated diphenyl oxide. For example, it is now possible to prepare reaction-derived decabromodiphenyl oxide of a purity of at least about 99%. Indeed, it is deemed possible to prepare reaction-derived products that contain at least about 99+% DBDPO and that contain amounts of nonabromodiphenyl oxide not exceeding 0.5%, preferably 0.3% or less, more preferably, no more than about 0.1%, and even more preferably no more than about 0.05%. Such products can be said to be “reaction-derived” since they are reaction determined and not the result of use of downstream purification techniques, such as recrystallization, chromatography, or like procedures. In other words, the products are of high purity.

In the various embodiments of this invention, the feeds of (i) to the refluxing bromine-Lewis acid catalyst-containing reaction mixtures can be diphenyl oxide (DPO) itself or one or a mixture of partially brominated diphenyl oxides formed by brominating diphenyl oxide with bromine in the absence of a catalyst. Such individual products and mixtures thereof can be used as feeds in the practice of this invention. The partially brominated DPO, which can be used as the feed in the practice of this invention, typically contains in the range of about 0.5 to about 4 atom(s) of bromine per molecule of DPO. Somewhat higher amounts of uncatalyzed ring-bromination of DPO can be accomplished under pressure, e.g., perhaps up to, say 5 or possibly even 6 atoms of bromine per molecule, by conducting the uncatalyzed reaction under pressure, or by use of a catalyst and such partially brominated DPO products or mixture can be used as feeds in the practice of this invention. In all cases, prior to its use as the feed of (i) to the refluxing bromine containing Lewis acid bromination catalyst, the hydrogen bromide coproduct should be removed from the partially brominated DPO feed or at least the amount of residual hydrogen bromide coproduct in the partially brominated DPO should be substantially reduced.

The DPO and/or partially brominated DPO can be fed as solids, but preferably the feed is in molten form or as a solution in an organic solvent such as methylene bromide or bromoform, and/or in liquid bromine. To prevent freeze up in the feed conduit, DPO is desirably fed at a temperature in the range of at least of 28 to 35° C. Higher temperatures can be used if desired or needed.

The bromine as fed to the reactor is desirably in the liquid state. It should be free of HBr or if HBr is present therein the amount should be at a minimum, preferably no more than about 100 ppm. Also, the amount of water in the bromine, if any, should be at a minimum, say, no more than about 10 ppm (wt/wt).

Excess bromine is used in the Lewis acid catalyzed bromination reaction. Typically, the reaction mixture will contain in the range of at least about 14 moles of bromine per mole of DPO to be fed thereto, and preferably, the reaction mixture contains in the range of about 16 to about 25 moles of bromine per mole of DPO to be fed thereto. It is possible to use more than 25 moles bromine per mole of DPO but this offers no advantage. When the feed is partially brominated DPO, enough bromine should be present to provide in the range of about 4 to about 12 moles of excess bromine over the amount required to perbrominate the partially brominated DPO. When the feed is a mixture of DPO and partially brominated DPO, the amount of excess bromine should be enough to provide a corresponding excess over the amounts sufficient to perbrominate the DPO and the partially brominated DPO.

Typically the refluxing temperature of bromine at atmospheric or slightly elevated pressures is in the range of about 57 to about 59° C. but when operating at higher elevated pressures somewhat higher temperatures are used in order to maintain a refluxing condition.

If desired, a suitable solvent can be included in the reaction mixtures. This can be advantageous in that one can have a higher reaction temperature and possibly a lower HBr concentration in the bromine thereby giving higher purity DBDPO. Among such solvents are methylene bromide and bromoform.

Various iron and/or aluminum Lewis acids can be added to the bromine to serve as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in a liquid phase of refluxing bromine. For example, one or more of the chlorine atoms of the aluminum chloride may possibly be replaced by bromine atoms. Other chemical changes are also possible. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

After all DPO and/or partially brominated DPO is added, the reaction mixture can be kept at reflux for a suitable period of time to ensure completion of the perbromination to DBDPO and to provide extra time for removal of hydrogen bromide from the reactor. A period of up to about 8 hours can be used.

Termination of the bromination reaction is typically effected by deactivating the catalyst with water and/or an aqueous base such as a solution of sodium hydroxide or potassium hydroxide.

### Gas Chromatographic Procedure

The gas chromatography is on a Hewlett-Packard 5890, series II, with Hewlett-Packard model 3396 series II integrator, the software of which is that installed with the integrator by the manufacturer. The gas chromatograph column used is an aluminum clad fused silica column, Code 12 AQ5 HT5 (Serial number A132903) obtained from SGE Scientific, with film thickness of 0.15 micron. The program conditions are: initial start temperature 250° C., ramped up to 300° C. at a rate of 5° C./min. The column head pressure is 10 psig (ca. 1.70×10⁵ Pa). The carrier gas is helium. The injection port temperature is 275° C. and the flame ionization temperature is 325° C. Samples are prepared by dissolving ca. 0.1 g in 8-10 mL of dibromomethane. The injection size is 2.0 microliters.

### DBDPO Products and Flame Retardant Usage

The DBDPO products formed in processes of this invention are white or slightly off-white in color. White color is advantageous as it simplifies the end-users task of insuring consistency of color in the articles that are flame retarded with the DBDPO products.

The DBDPO products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The DBDPO products of this invention can be used in textile applications, such as in latex-based back coatings.

The amount of a DBDPO product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any give application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % DBDPO product of this invention. Masterbatches of polymer containing DBDPO, which are blended with additional amounts of substrate polymer, typically contain even higher concentrations of DBDPO, e.g., up to 50 wt % or more.

It is advantageous to use the DBDPO products of this invention in combination with antimony-based synergists, e.g., Sb₂O₃. Such use is conventionally practiced in all DBDPO applications. Generally, the DBDPO products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPO products of this invention, e.g., plasticizers, antioxidants, fillers, pigments, UV stabilizers, etc.

Thermoplastic articles formed from formulations containing a thermoplastic polymer and DBDPO product of this invention can be produced conventionally, e.g., by injection molding, extrusion molding, compression molding, and the like. Blow molding may also be appropriate in certain cases.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

Each and every patent or publication referred to in any portion of this specification is incorporated in toto into this disclosure by reference, as if fully set forth herein.

That which is claimed is:

1. In a process for preparing reaction-derived decabromodiphenyl oxide of high purity, which process comprises feeding diphenyl oxide and/or partially brominated diphenyl oxide into a reactor containing a refluxing reaction mixture comprising (i) an excess of bromine and (ii) a catalytic quantity of Lewis acid bromination catalyst, and substantially concurrently removing hydrogen bromide coproduct from the reactor in a sufficient amount to form a reaction-derived decabromodiphenyl oxide product of high purity, the improvement which comprises:
A) substantially concurrently feeding at least two separate feeds into said reactor, one of said separate feeds being bromine and the other, or another, of said separate feeds comprising said diphenyl oxide and/or partially brominated diphenyl oxide; or
B) substantially concurrently feeding at least two separate feeds into a small mixing zone, one of said separate feeds being bromine and the other, or another, of said separate feeds comprising said diphenyl oxide and/or partially brominated diphenyl oxide, followed within 2 seconds or less by feeding the resultant mixture into said reactor.

2. The improvement as in claim 1 wherein the improvement is the improvement of A).

3. The improvement as in claim 1 wherein the improvement is the improvement of B).

4. The process as in claim 1 wherein a flow of inert gas is passed through the reactor during the feed and/or upon completion of the feed.

5. The process as in claim 1 wherein a flow of bromine vapor is passed through the reactor during the feed and/or upon completion of the feed.

6. The process as in claim 1 wherein the reaction mixture is subjected to fractionation in a fractionation column during the feed and/or upon completion of the feed.

7. The process as in claim 1 wherein the reaction period provided in the reactor is inversely related to the temperature at which the refluxing is occurring.

8. The process as in claim 1 wherein the reaction mixture is maintained at about atmospheric pressure.

9. In a process of preparing reaction-derived decabromodiphenyl oxide of high purity, which process comprises maintaining a substantially continuous, inversely related time-temperature feed of diphenyl oxide and/or partially brominated diphenyl oxide to a reactor containing a refluxing reaction mixture comprising (i) an excess of bromine and (ii) a catalytic quantity of Lewis acid bromination catalyst, and substantially concurrently reducing the concentration of hydrogen bromide coproduct dissolved in the liquid phase of the reaction mixture so that reaction-derived decabromodiphenyl oxide product of high purity is formed, the improvement which comprises:
A) substantially concurrently feeding at least two separate feeds into said reactor, one of said separate feeds being bromine and the other, or another, of said separate feeds comprising said diphenyl oxide and/or partially brominated diphenyl oxide; or
B) substantially concurrently feeding at least two separate feeds into a small mixing zone, one of said separate feeds being bromine and the other, or another, of said separate feeds comprising said diphenyl oxide and/or partially brominated diphenyl oxide, followed within 2 seconds or less by feeding the resultant mixture into said reactor.
